(19) United States
(12) Patent Application Publication
(10) Pub. No.: US 2005/0038363 A1
(43) Pub. Date: Feb. 17, 2005

(54) Foldable orthosis night splint with ortho-wedge

(76) Inventor: William S. Stano, Boise, ID (US)

Correspondence Address: FRANK J. DYKAS;DYKAS, SHAVER & NIPPER, LLP, P.O. BOX 877, BOISE, ID, 83701-0877, US

(21) Appl. No.: 10/641,886

(22) Filed: Aug. 14, 2003

(51) Int. Cl. A61F 5/00

(52) U.S. Cl. 602/5

(57) ABSTRACT

A low cost foldable orthosis for the treatment of foot and ankle conditions including plantar facitis and tendonitis, the orthosis is a foldable molded portion manufactured in a variety of incremental sizes, having a generally U-shaped cross-sectional configuration and a flat foot bed, covered by a soft fabric covering, and using a removable and interchangeable foot bed wedge insert permitting the angle of dorsiflexion, plantar flexion, inversion and eversion to be varied.

### BACKGROUND OF THE INVENTION

Field of the Invention. The present invention relates to orthotic devices and more specifically to a foldable night splint for treating and facilitating the treatment of the pain in the foot and heel caused by contracture of the plantar fascia and/or the Achilles tendon, treatment of hip ailments, and post-surgery treatment of the foot.

Background. Simply put, the human foot takes the brunt of the impact of every step experienced by an individual. It is also likely that the single largest source of complaint for foot ailments is related to heel pain. One source of heel pain commonly observed is due to a condition known as recalcitrant plantar facitis. Plantar facitis occurs in the plantar fascia, a fibrous membrane disposed longitudinally across the bottom of the foot. The plantar fascia is attached at the heel bone, more specifically to the inner tubercle of the os calcis. The plantar fascia becomes broader and thinner as it extends longitudinally across the bottom of the foot, eventually dividing near the heads of the metatarsal bones into five processes, one for each of the five toes.

The strongest ligament in the body is the plantar fascia, a fibrous band of tissue that starts on the bottom surface of the heel bone and extends forward on the bottom of the foot to just behind the toes. Its function is to protect the softer muscles and tissues of the bottom of the foot from injury, as well as to help maintain the integrity of the foot structure itself. If the fascia becomes stretched or strained, the arch area, as well about the heel bone, become tender and swollen. This inflammation is called plantar facitis and is painful from the heel throughout the arch up into the Achilles tendon. These patients usually have tight and inflexible heel cords, a condition that is referred to as Achilles tendon tightness.

When the heel cord is tight, it causes compensation in the foot with over-pronation of the foot during weight bearing. The pain is consistently worse when you first get up in the morning and at the end of the day. The pain usually lurks in the heel pad and may include the arch ligament. The natural tendency is to ignore the symptoms of the pain at first.

Heel pain like plantar facitis is often times caused by contracture of the Achilles tendon and the plantar fascia, which can occur at night during sleep or during daytime inactivity. The Achilles tendon is the strongest and thickest tendon in the human body. The Achilles tendon begins at or about the middle of the posterior side of the leg extending downward towards the heel, narrowing as it progresses towards its point of insertion at the posterior surface of the os calcis. When an individual is standing, walking, running or even sitting in a position in which the feet are in contact with the floor or other surface, both the plantar facia and the Achilles tendon are extended to varying degrees depending of course on the nature and intensity of the activity. During sleep, an individual has a natural tendency to plantar-flex the ankle joint beyond the position which is normal during walking, standing or sitting with one's feet on the floor. Plantar flexion is when the bottom of the foot is extended so as to form an angle with the lower leg of greater than 90°. Dorsiflexion is the opposite motion; when the foot is moved to a position in which the bottom of the foot forms an angle with the lower leg of less than 90°, this is dorsiflexion. As a result of plantar flexion during the night, the plantar facia and the Achilles tendon contract from their size and dimension normal to the walking, standing or sitting positions. Upon arising, the plantar facia and the Achilles tendon are once again extended and stretched when the feet and ankles resume a normal position associated with walking or standing. Typically, it is when an individual arises following sleep or a period of extended recumbancy that the effects of heel pain associated with plantar facitis, with or without the associated Achilles tendon contracture, are observed. In a significant number of cases, the pain has been described as substantial.

Various theories explain the constant pull of the plantar fascia at the insertion of the heel bone. The plantar fascia and intrinsic muscles can cause spurs or tearing of the fascia at the insertion. With continued pull, subperiosteal bleeding can produce calcification leading to new bone. Other theories are constant stress of the fascia with excessive stress at the insertion forming new connective tissue with the tissue going from fibrocartilaginous tissue to cartilaginous to bone. A reference to the thickening of the plantar ligaments is found as early as 1859 in a dissection of a flat foot by Dr. Wood.

In various types of occupations, sedentary work may produce atrophy and degeneration of the shock absorption ability of the heel's fat pad. Occupations which produce over use of tissue, which is stressed beyond its physiologic limits, such as working at a factory machine or the static loading exposure of welding, may also cause fat pad atrophy and degeneration from long, unnatural hours of standing on hard surfaces owing to the degeneration of the plantar fascia.

Plantar facitis is a condition characterized by tenderness located at or near the point at which the plantar fascia attaches to the heel bone, or the os calcis. This condition has been traditionally treated in a number of ways, including non-steroidal anti-inflammatory medicines, cortisone injections, shoe modifications, physical therapy, and even surgery.

Plantar facitis is referred to in a book in 1915 by Dr. Scholl as “policeman's heel.” Reference can be found in literature on heel pain before 1900. Authors writing about the conditions affecting the foot referenced it as pain of various courses from systemic disease to pain related to the plantar fascia. In 1860, Zacharie discussed a condition affecting the heel in which patients had greater pain in the morning than after standing and walking for one or two hours. In 1900, Plettner noticed inferior heel spurs on patients' radiographs. After that, many theories were put forth on the cause of heel pain and plantar facitis and the amount of references in the literature had more prevalence in this time. In 1915, Dr. Scholl indicated that painful heel pain was usually accompanied by flat foot, giving us the revelation that was correlation between pronation and painful heels.

The earliest records reviewed found treatment for heel pain was in a 1915 article by Waechter and Sonnenschein in which they used felt aperture pads for the treatment of painful heel pain. Dr. Scholl in 1915 advocated the use of a metal orthotic called the Trispring. Metal was placed into the arch to support it and prevent elongation of the arch and a leather top was applied over the metal. Dr. Carl Bergman states that in his orthopedic lecture notes taken at the Illinois College of Chiropody in 1919 suggests the use of a sponge heel pad in the shoe for the local relief of heel pain.

Favorable results for the treatment of plantar facitis have been observed in a study that employed night splints in connection with other non-surgical therapeutic measures to treat this condition. See Wapner and Sharkey, “The Use Of Night Splints For Treatment Of Recalcitrant Plantar Facitis,” Foot and Ankle Vol. 12, No. 3, December 1991. The night splint consists, essentially, of a boot-like structure that is strapped to a patient's lower leg and foot, holding the foot relative to the lower leg in a position such that the ankle joint is held in slight dorsiflexion. In so doing, both the plantar fascia and the Achilles tendon are slightly extended and are not allowed to contract during the night. The use of night splints, together with the variety of other elements of treatment including anti-inflammatory medications, physical therapy, and foot cushions for use during the daytime, has proved beneficial in the treatment of plantar facitis.

It is desirable to have an orthosis that has the possibility of inducing inversion or eversion of a patient's foot. Inversion is when the bottom of the foot, the plantar surface, faces more toward the midline of the body. Eversion is motion of the foot in which the plantar surface of the foot is tilted so as to face further away from the midline of the body.

The splints described in the Wapner-Sharkey article consisted of a custom molded ankle-foot orthosis constructed of polypropylene. The authors of that article approximate the cost of each splint at $200.00. See Wapner/Sharkey at pages 135 and 136.

It is suggested that the relatively high price of the splints used in the Wapner-Sharkey study is due in part to the custom molding required to form the splint to conform to the patient's anatomy. Additionally, a custom molded orthosis can be used by only one patient.

Various other splints are advertised for treatment of plantar facitis that also typically consist of a molded splint or a combination of molded plastic and metal framework, with the dorsiflexion set at 5°.

Although similar in appearance to foot and ankle casts, also called walking casts, a night splint for the treatment of plantar facitis is only superficially similar to a walking cast. A foot or ankle cast is made so that the force vector of the patient's weight passes vertically through the cast and the patient's leg when he/she is standing. In the medical industry, no walking casts are made that do not place the bottom of the patient's foot at a 90° angle to the patient's leg, which is consistent with a vertical force vector. Thus, no walking casts are built to induce and maintain dorsiflexion or plantar flexion. In addition, a walking cast is made to provide the patient with a weight-bearing region forward of the heel, on which the weight of the body is placed when walking, and from which the patient can pivot forward when taking the next stride. The bearing and pivoting structure can be a rounded knob under the mid region of the foot, or it can be a rounded surface which covers the bottom of the cast from heel to toe. A walking cast may also have a cushioning region directly under the heel to absorb some of the shock of walking.

Walking casts are not made to wear in bed at night, and are not made to induce a stretching effect on tendons. They are made to provide support to healing ankle and foot joints and bones, and to control the motion of these healing joints and bones while healing takes place.

To treat plantar facitis, it is necessary to use considerable force to counteract the strong muscles and tendons of the lower leg and foot. If this force is applied improperly, pressure points can result, thus causing discomfort and complications for some patients.

Some patients have reduced blood circulation or sensation in the feet, such as patients with diabetes, vascular insufficiency, polio, stroke, trauma, or neurological problems. In such patients, if they need to use a night splint for treatment of plantar facitis, it is important to minimize the pressure points exerted by the night splint on the patient's foot, while still exerting the necessary force on the foot and lower leg structure. The night splint must also not bruise or scratch the collateral leg during sleep, must not soil or tear bedding, and must be compatible with a sleeping partner. Walking casts are not designed to accomplish these objects.

Another ailment for which a night splint is needed is calcaneal apophysitis. This is typically a problem which presents in juveniles. It is basically a case of the bones of the leg and foot growing faster than the connective tissue, such as the plantar fascia and Achilles tendon, and the heel bone is immature and somewhat soft. These two tendons are put under strain and cause heel pain. Treatment of calcaneal apophysitis has proven to be very successful using a night splint. The night splint prevents foot drop during sleep, and helps lengthen the two involved tendons.

Paratendon tendonitis is another condition for which a night splint is needed for successful treatment. The paratendon is a thin sheath-like covering of tendons. The lining of this structure can become inflamed, and require nighttime stabilization to immobilize the foot and lower leg and treatment.

Achilles tendonitis is another condition for which a night splint is needed for successful treatment. Achilles tendonitis can result from overuse of the tendon in sports activities, and can also result from a number of inflammatory diseases, of which rheumatoid arthritis is one. Use of a night splint is an effective treatment for this ailment, since immobilizing the Achilles tendon without allowing night drop or contracture of the tendon is the best treatment.

Another area where a night splint is needed is after various surgeries on the hip. After hip replacement, for instance, it is desired that the involved hip joint remain absolutely immobile. What is needed is a device that immobilizes one or both feet and lower legs, so that the hip joint is not moved.

Another situation that requires the use of a night splint is when surgery has been performed on tendons in the foot. If the tendons worked on are on the medial side of the foot, it is desirable for the foot to be held in an inverted position (with the plantar surface facing toward the midline of the body), which relieves strain on the affected tendons. If the tendons worked on are on the lateral side of the foot, an everted position is desirable.

Night splints function best when they can be used on a continuous ongoing basis, thus allowing the tendons to be appropriately stretched into a desired position. One problem that exists with walking casts and similar devices is that they are bulky and are difficult to store in a desired position. Another problem is that such devices have an upper portion and a lower portion that are configured in relatively fixed positions relative to one another. This results in many of the devices shown in the prior art being large and bulky and not easily stored for transport or storage. As a result of this phenomenon, many times the individuals who should utilize such devices, fail to do so.

Accordingly, it is an object of the invention to provide an orthosis which is suitable for use on a patient's foot and lower leg during the night, as a night splint for the treatment of plantar facitis, Achilles tendon problems, hip immobilization, and post-surgery treatment of the foot. The orthosis needs to hold the foot in a generally dorsiflexed position with adjustments available for holding the plantar surface of the foot from 90° to 75° from the longitudinal axis of the lower leg.

It is also an object of the invention to provide an orthosis that secures the foot in a dorsiflexed position, and which secures the heel in a floating heel cup.

It is another object of the invention to provide an orthosis that is anatomically designed to be close fitting, in order to provide support and to reduce pressure points.

It is a further object of the present invention to provide a splint or orthosis that may be employed in treating plantar facitis, contractures of the Achilles tendon, and other tendinous structures of the foot, such as the flexor tendons for the feet and ankles that would be lower in cost and more versatile than the presently available alternatives.

It is also an object of the present invention to provide an orthosis having removable and interchangeable foot wedge inserts that will permit the angle of dorsiflexion to be varied, as well as the angle of inversion and eversion. This allows certain therapeutic advantages. Additionally, the ankle joint may be extended beyond the neutral position, (dorsiextension) or rotated medially or laterally, for other various therapeutic uses merely by substituting foot bed inserts.

Finally, an objective of the present invention is to provide a low cost orthosis that consists of foldable molded portions that is manufactured in a variety of incremental sizes, i.e. extra small, small, medium, large and extra large, which may be used by a variety of patients obviating the need for custom molding. These foldable portions have a shorter lower leg portion as compared to other prior art devices and is foldable so as to allow the lower leg portion to be stored in a location such as a standard size shoebox. The portion is also extendible from the folded position to another position that is configured to form a desired shaped orthotic and to be quickly and efficiently moved into another desired position depending upon the necessities of a user.

Additional objects, advantages and novel features of the invention will be set forth in part in the description which follows and in part will become apparent to those skilled in the art upon examination of the following or may be learned by practice of the invention. The objects and advantages of the invention may be realized and attained by means of the instrumentalities and combinations particularly pointed out in the appended claims.

### SUMMARY OF THE INVENTION

According to the present invention, the foregoing and other objects and advantages are attained by a device for treating plantar facitis that includes an upper portion and a lower portion. Both sections are generally U-shaped in cross-section. The lower section has a generally flat foot bed portion. The upper and lower sections are pivotally connected one to another so that the upper and lower sections are foldable from a first position wherein the lower section extends from the generally upright upper portion at an angle of less than 90° to a second position wherein the upper portion is generally folded over upon the lower foot bed section of the device. The upper and lower sections are structurally configured to be locked or maintained in a desired position by the structure of the upper and lower sections themselves.

The upper section is configured to generally conform to the lower portion of the human leg, and the lower section is configured to receive a bottom surface of a foot attached to a human leg. A removable wedge foot bed insert is included as part of the device and it is configured to be received in the foot bed portion of the lower portion. The wedge is typically inclined from a heel portion to a toe portion and thus forms an inclined foot bed that prevents plantar flexion and promotes dorsiflexion. A securing mechanism is also included as part of the device and is used to secure a patient's foot in the device for treating plantar facitis. The securing mechanism is flexible in at least one area above the foot bed to allow for adjustable degrees of dorsiflexion while preventing plantar flexion past the fixed angle of the inclined foot bed.

Removable wedge foot bed inserts result in foot beds that are less than 90° in relation to the upper portion. An angle between 75° and less than 90° has been found to be an optimal range. The device for treating plantar facitis can be made from a variety of sizes of portions. The combination of variously configured portions and variously configured wedge foot bed inserts can be combined to achieve orthotics having desired shapes, sizes and other characteristics. In some embodiments, various desired positions of the foot can be further achieved by a rear heel cup that is positioned to receive and hold a various foot positioning devices, which can provide both cushioning and support to the foot being held within the device.

The present invention may be formed in various sizes and shapes according to the traditional shoe sizes that are required and utilized in the prior art. The sizes correspond to U.S. shoe sizes, as described in the Description Of The Preferred Embodiments section contained herein. It is important that the correct size be selected so that the shape of the device is properly proportioned to the length of a person's lower leg. By properly sizing the device, the heel can be secured in a floating position so that it does not touch the foot bed. This device can also be configured with a removable wedge foot bed insert which is higher on one side of the removable wedge foot bed insert than on the other side. This results in inversion or eversion of the patient's foot when placed in the device. A range of greater than 0° and inclusive of 15° has been found to be an optimal range for inversion or eversion.

Another aspect of the invention is a method for treating plantar facitis. The method consists of securing the upper portion of the present invention to the lower posterior portion of the leg and foot of a patient. The method also includes inserting a removable wedge foot bed insert that is inclined from a heel portion of a foot bed to a toe portion of the foot bed, and which forms an inclined foot bed in the lower portion. This inclined foot bed prevents plantar flexion of the foot, and induces dorsiflexion of the foot.

In some embodiments, the lower portion that is pivotally attached to the upper portion may then be modifiably locked into various positions to achieve a desired amount of flexion of the foot with regard to the lower portion of the leg. These portions are secured to the leg and foot by means of a securing mechanism that is flexible in at least an area above the foot bed to allow for adjustable degrees of dorsiflexion while preventing plantar flexion past the inclined foot bed.

Another aspect of the invention is a method for treating plantar facitis whose steps include inserting a removable wedge into a foot bed portion of a foldable night splint, the foldable night splint having an upper and a lower section held in a hinged interconnection. Both the upper and the lower sections have a generally U-shaped cross-section. The upper section is designed to generally conform to the lower portion of a human leg. The lower section is designed to receive a bottom surface of the patient's foot, as well as to hold a desired shaped wedge within the device. The wedge is also configured to support a bottom surface of a foot, and is inclined from a heel portion to a toe portion. The wedge, therefore, forms an inclined foot bed which prevents plantar flexion. Another step in the method is securing the upper section to the lower posterior portion of a leg and foot of a patient using a securing mechanism that is flexible in at least one area above the foot bed to allow for adjustable degrees of dorsiflexion, while preventing plantar flexion past the desired positioning of the inclined foot bed.

Another aspect of the invention is that the foldable orthosis includes a generally curved portion that is configured to receive a portion of a lower leg and that also is configured to maintain the lower portion of the device in a desired amount of dorsiflexion with regard to the lower portion. In addition to this feature, the angle between the upper and lower portions of the device can be modified and held in a desired orientation by the insertion of spacing devices between the heel locking portion of the upper and lower portions. These spacing devices can be utilized to vary the positioning of the upper and lower heel portions and thus the designated amount of flexion which a patient's foot can be oriented.

The lower portion of the orthotic has a heel section and a toe portion, and extends at an angle of less than 90° from the upper section when the foldable portion is fully flexed and extended. When this device is fully flexed and extended, this configuration is locked into a static alignment. Depending upon the necessities of the various users, varying degrees of extension and flexion for locking the device may be utilized. The lower section also has a generally flat foot bed portion, with the heel portion narrower than the toe portion and designed for close anatomical fit with the heel of a human patient. The lower section is configured to contain both a foot and a foot wedge bed. The lower section also has sides that extend with sufficient height so as to provide the designated amounts of support to the foot of the individual that is utilizing the device.

The upper section is configured for close and anatomically conforming to the lower posterior portion of the human leg. The upper section also includes a sagittal concavity that conforms to the human leg and maintains the human heel in a floated position from the flat foot bed. This floated position of the heel is achieved by securing the leg in the upper portion against the sagittal concavity. The upper portion has a length that corresponds with the distance from the patient's heel to a point below the thickest portion of the gastrosoleus muscles. The length of this upper portion is designed to provide optimal support to the leg and muscles involved and to reduce pressure points. When extended the upper and lower portions of the device are configured to substantially cover the heel of the individual utilizing the device.

The lower portion is configured to be about the same length as the foot of the patient. Typically, the upper portion is relatively shorter than the lower portion. The lower portion is designed to receive a removable foot bed insert. In the preferred embodiment, the upper and lower portions are covered with a soft covering that cushions the leg of the user from contact with the generally inflexible materials that make up the upper and lower sections of the device. This configuration of the device also includes a removable wedge foot bed insert that is typically inclined from a heel portion to a toe portion of the foot bed. It therefore forms an inclined foot bed which prevents plantar flexion of the foot. The removable wedge foot bed has a cushioning top surface that is soft and flexible, and also contains a semi-rigid material to which the cushioning top surface is attached. The device also includes a soft jacket that covers the inside and outside surfaces of the upper and lower portions to which the securing device is attached. The securing device is flexible in at least one area above the foot bed to allow for adjustable degrees of dorsiflexion, while preventing plantar flexion past the inclined foot bed. Removable wedge foot bed inserts can be designed to result in a foot bed orientation from less than 90° to 75°. In addition, to the removable foot wedges providing desired foot and lower leg positioning, the inclusion of spacers together with the foldable lockout joint allow the devices to be adjustably locked into desired positions and configurations.

In another aspect of the invention, the invention consists of a method for treating plantar facitis in a human patient that includes the steps of inserting a removable wedge into a foot bed portion of a foldable orthotic having an upper and a lower portions both portions having a generally U-shaped cross-section, with the lower portion also having a heel portion and a toe portion, and being narrower in the heel portion than in the toe portion to facilitate a close anatomical fit to a human foot. The lower portion extends at an angle of less than 90° from the upper section when the two sections are fully extended to a lock out position and has a generally flat foot bed portion. The upper section is designed for close anatomical conformance to the lower portion of a human leg below the thickest part of the patient's gastrosoleus muscle. This portion is covered with a soft covering on its inside and outside surface. The wedge is typically inclined from a heel portion of the foot bed to a toe portion of the foot bed, and thus forms an inclined foot bed that prevents plantar flexion.

Another step of the method is securing the device to the lower posterior portion of a leg and foot using a securing mechanism which is flexible in at least an area above the foot bed. This flexibility allows for adjustable degrees of dorsiflexion while preventing plantar flexion past the inclined foot bed. Another step of the method is requiring the patient to wear the foldable orthotic, the soft covering and the wedge, secured by the securing mechanism to the lower posterior portions of the leg and the foot, while in a reclining position which can occur during sleep or at other times.

In another aspect of the invention, the invention is a method for preventing hip movement in a human patient and includes the steps of securing the upper portion to the lower limp limb of a patient. The portion having an inner and an outer surface and an upper section and a lower section. Both sections have generally U-shaped cross-sectional shapes. The lower section includes a heel portion and a toe portion. The heel portion being narrower than the toe portion for close anatomical fit to a human foot. The lower section extends at an angle approximately less than 90° from the upper section and has a generally flat foot bed portion. The upper section is designed for close anatomical conformance to a lower portion of a human leg and, when worn by a patient, extends from the heel of a patient to a portion upward then to a point below the gastrosoleus muscle. The lower section is configured to receive a bottom surface of the patient's foot. The upper and lower portions are each covered with a soft covering on at least their inside surface and may be covered on its outside surface in addition.

The next step in the process is securing the lower portion of the device to the lower portion of the leg and the foot of a human patient. This is accomplished through the use of a securing mechanism that is flexible in at least an area above the foot bed to allow for adjustable degrees of dorsiflexion while preventing plantar flexion past the inclined foot bed. Another step in the method is placing the portion with the leg and foot of a patient in a stabilizing cradle and securing the portion in a fixed position in the stabilizing cradle with a means of attachment. This means of attachment can be Velcro® straps, hook and loop fasteners or other conventional means of securement.

The next step of the method is requiring the patient to wear the device and the soft covering. The device is secured to a patient's leg by a securing mechanism to the lower posterior portion of the leg and the foot and the portion attached to the stabilizing cradle with a means of attachment while in a reclining position. Optionally, a wedge may be used in the orthosis.

The method and apparatus of the invention, using the foldable orthotic holds the foot at an angle of less than 90° to the leg, prevents plantar flexion of the foot and promotes dorsiflexion of the foot without applying pressure to the heel. The orthosis can also be used for post surgery treatment of a foot, leg, and/or hip, to relieve pressure on the area that has been operated on and to immobile areas requiring such immobilization.

Further, the purpose of the foregoing abstract is to enable the United States Patent and Trademark Office and the public generally, and especially the scientists, engineers, and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

Still other objects and advantages of the present invention will become readily apparent to those skilled in this art from the following detailed description wherein I have shown and described only the preferred embodiment of the invention, simply by way of illustration of the best mode contemplated by carrying out my invention. As will be realized, the invention is capable of modification in various obvious respects all without departing from the invention. Accordingly, the drawings and description of the preferred embodiment are to be regarded as illustrative in nature, and not as restrictive in nature.

### BRIEF DESCRIPTION OF THE DRAWINGS

FIG. 1 is a perspective view showing the ankle foot orthosis.

FIG. 2 is a perspective representational view of the upper and lower portions of the ankle foot orthosis when properly connected.

FIG. 3 is a side representational view of the embodiment shown in FIG. 2 in a folded position.

FIG. 4 is a detailed view of the connection between the upper and lower sections of the present invention.

FIG. 5 is a cut away side view of the invention shown in FIG. 1.

FIG. 6 is an end view of the embodiment shown in FIG. 3.

FIG. 7 is a side plan view of a 10° removable foot bed wedge insert.

FIG. 8 is a side plan view of a 5° removable foot bed wedge insert.

FIG. 9 is a dorsal or bottom anatomical plan view of a human foot.

FIG. 10 is a side view of a human foot and a removable foot bed wedge insert.

FIG. 11 is a representational view of a removable wedge which when used with the orthosis would result in inversion or eversion of the patient's feet.

FIG. 12 is a front view of a human foot and a removable foot bed wedge insert in everted configuration.

FIG. 13 is a front view of a human foot and a removable foot bed wedge insert in inverted configuration.

FIG. 14 is a perspective view of an ankle foot orthosis used in conjunction with a stabilizing cradle.

### DESCRIPTION OF THE PREFERRED EMBODIMENTS

While the invention is susceptible of various modifications and alternative constructions, certain illustrated embodiments thereof have been shown in the drawings and will be described below in detail. It should be understood, however, that there is no intention to limit the invention to the specific form disclosed, but, on the contrary, the invention is to cover all modifications, alternative constructions, and equivalents falling within the spirit and scope of the invention as defined in the claims.

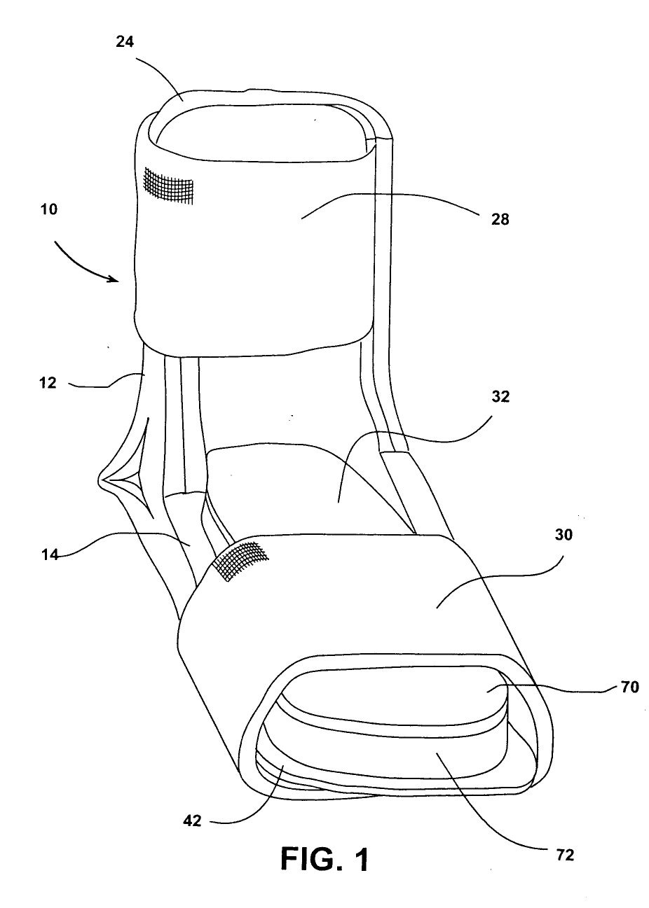

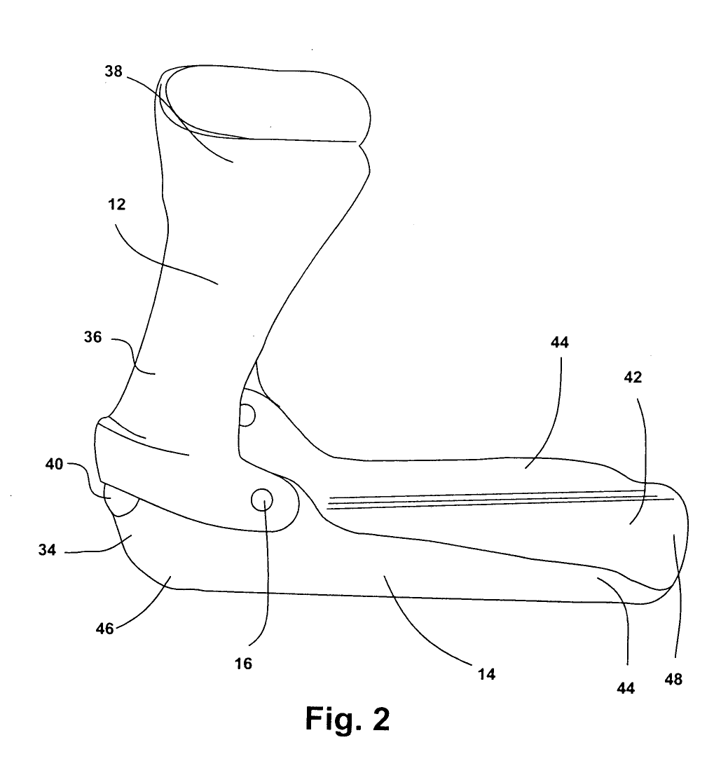

Referring to FIGS. 1 through 6 and 14, the ankle foot orthosis of the present invention is shown to advantage. FIG. 1 shows the ankle foot orthosis 10 which is configured for connection with a lower leg and foot of a patient. As shown in FIGS. 1-6, ankle foot orthosis 10 consists of an upper portion 12 that is pivotally connected to a lower portion 14 through a hinge 16 (shown in FIG. 2). A fabric covering 24 covers the inside and outside surfaces of the upper and lower portions 12, 14. A removable wedge foot bed insert 32 is configured to provide a desired level of plantar and dorsiflexion to the individual. In addition, a lower leg attachment strap 28 and a foot attachment strap 30 provide means for attaching the device 10 to the foot and lower leg of a patient. The fabric covering 24 surrounds and covers selected portions of the inside and outside surfaces of upper and lower portions 12, 14.

In this preferred mode, removable wedge foot bed insert 32 is composed of two layers of foam, a soft-top layer 70, and a firm foam layer 72. In the preferred mode, soft top layer 70 is made of Sentinel Blue F-Cell MTL foam, a cross-linked polyethylene foam. However, another soft material could also be utilized. Firm foam layer 72 is preferably Sentinel White MTL F-Cell AW900, a cross-linked polyethylene foam. However, other materials could be utilized that provide support and resist deformation. In the preferred embodiment, removable wedge foot bed insert 32 fits the flat foot bed 42 of the lower portion 14. This means that removable wedge foot bed insert 32 is narrower at the heel portion 46 than at the toe portion 48. A variety of foot bed angles can be formed from removable wedge foot bed inserts 32 of varying angles. Other embodiments of the preferred mode may utilize removable foot bed inserts 32 which vary in thickness from one side to another, as will be discussed later.

Fabric covering 24 is provided with a lower leg attachment strap 28 and a foot attachment strap 30, which attach the device 10 to a patient's leg and foot with a plurality of hook type fasteners so as to provide easy attachment and detachment of the device from a desired location along the foot of a user. While hook type fasteners are shown, it is to be distinctly understood that other types of devices for fastening a device or means of fastening can be utilized, such as the use of straps and buckles, metal loops to pass straps through, and any conventional means of attachment.

Referring now to FIG. 2, the internal structure of the device 10 is shown. The portions of the device 10 are comprised of an upper portion 12, and a lower portion 14. These sections 12, 14 are pivotally connected to each other through a hinge 16. In the preferred mode, the portion 12, 14 are made up of plastic, although other materials could be utilized. In the preferred mode, lower portion 14 and the upper portion 12 are configured to extend apart from one another to reach and angle at their greatest degree of separation of approximately 80°. The upper portion 12 and lower portion 14 each have a cross-section that is generally U-shaped and are configured to accommodate the respective anatomical portions of the individual wearing the device as well as other items such as removable foot wedges and other such devices.

Upper portion 12 is shaped to closely follow the contours of the posterior of a patient's foot and lower leg. It contains a sagittal concavity 36 and a leg flare 38 that are configured to receive and hold desired portions of an individual's leg therein. These portions 36, 38 are also configured to maintain a desired amount of dorsiflexion upon the foot of the person wearing the device. The lower portion 14 is configured to follow the contours of the posterior and lower portions of a patient's foot. The lower portion has a heel pocket 34 that further provides a rear heel cup 40 and a foot bed 42, which is configured to receive an orthopedic wedge having a desired shape and orientation as well as the foot of an individual. In this preferred embodiment, when the upper and lower portions are fully extended the heel of the individual is fully enclosed. The foot bed 42 also has side pieces 44 that run along each side of the foot bed 42. Flat foot bed 42 has a heel portion 46 and a toe portion 48. The foot bed 42 is narrower at the heel portion 46 than at the toe portion 48 to accommodate the typical contour of a human foot.

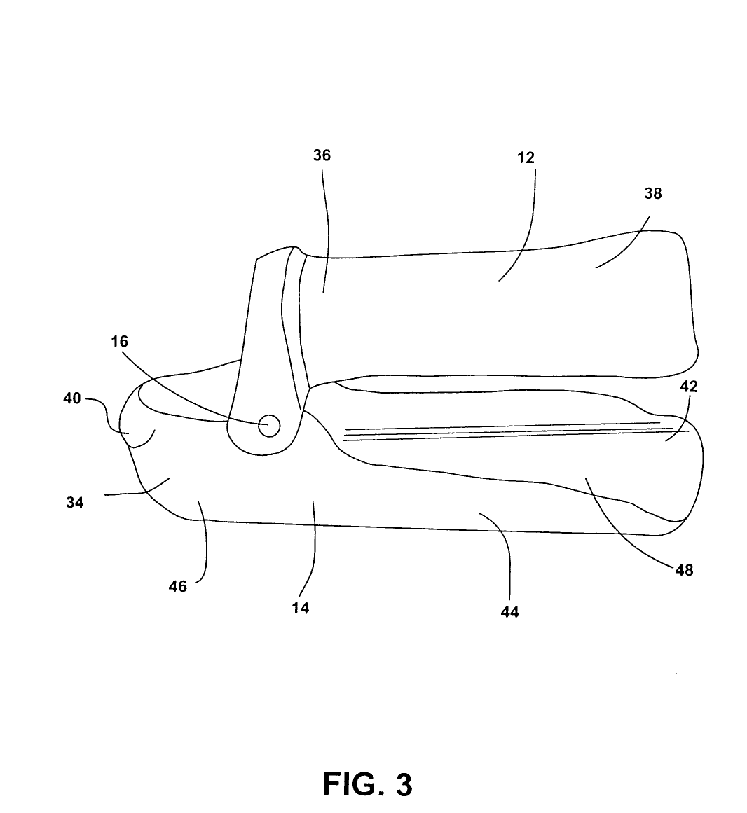

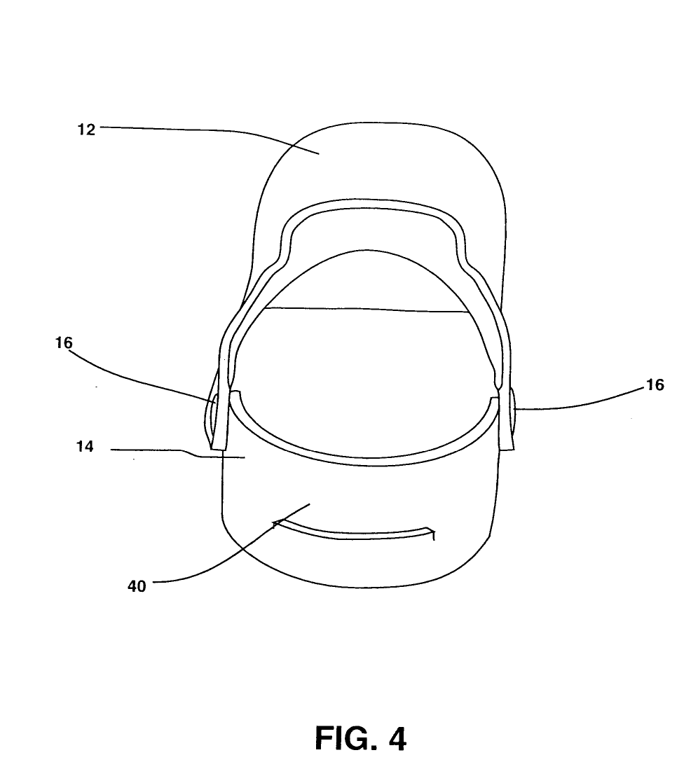

Near the hinge portion of the device 16, where the upper portion 12 and the lower portion 14 intersect, the upper and lower portions of the device are flared so as to provide a desired amount of space for the brace 10 to extend around the ankle protrusions of the patient. In addition a ridge 52 is formed within the upper portion 12 of the device so as to interact with the heel cup portion 40 so as to allow the upper 12 and lower 14 portions of the device to be locked in a designated position. This junction is a relatively static type of union that prevents a patient's foot from significant alteration from this designated position. A variety of spacers, preferably H-shaped rubber spacing blocks provide increased variations in the positioning of the upper and lower portions of the device. These spacing blocks function to limit the extending angle that the upper 12 and lower 14 portions of the device that the device may be extended to. Referring now to FIG. 3, a side view of the embodiment shown in FIG. 2 is shown in a folded, or compacted position. In this position, the device 10 can also be stored in a variety of containers, including, but not limited to, a standard shoe size box. This feature also allows the present device to be transported to a variety of locations and increases the likelihood that an individual will actually use the device to obtain the desired therapeutic effects. An end view of this embodiment is shown as FIG. 4.

FIG. 4 shows an end view of the preferred embodiment of the invention when the invention is in a folded position. In this position, the rim 52 which is defined within the upper portion 12 is not in contact with the heel cup portion 40. In this folded position, the device 10 is configured for placement and insertion with a storage container such as a standard sized shoe box.

The angle of extension between the lower portion 14 and the upper portion 12 can be varied by the placement of generally H-shaped spacers between the upper 12 and lower 14 portions. These spacers can be inserted between the upper 12 and lower portions 14 so as to hold the device in a desired position and orientation. When these spacers are used in such a manner the positioning and orientation of the upper 12 and lower 14 portions of the device can be modified and oriented so as to achieve a desired amount of dorsiflexion in the foot of the user. In addition to modifying the angle of extension between the upper 12 and lower 14 portions of the device 10, the angle of dorsiflexion for a foot can also be variously modified by varying the wedges within the device as well as the amount of curvature within the upper portion 12 of the device.

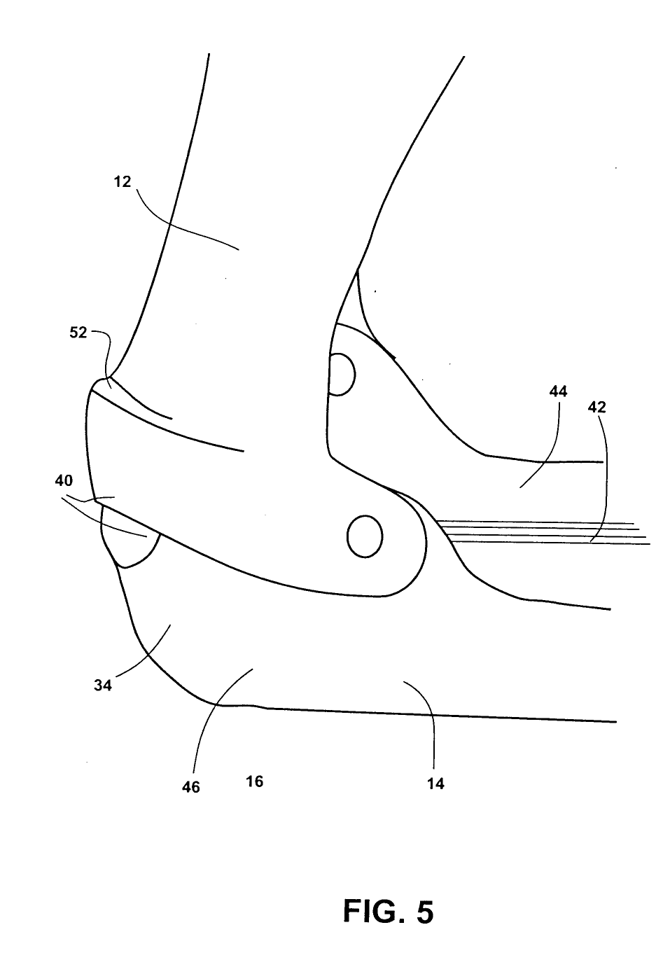

FIG. 5 shows a detailed side view of the embodiment of FIG. 2 wherein the connection between the upper and lower sections 12, 14 are shown to advantage. The maintenance of the upper section 12 in a desired position with regard to the lower portion 14 is accomplished by the intersection of a ridge 52 that is located within the upper portion of the device 12. This ridge 52 is configured to intersect with a portion of the lower portion 14 so as to hold the upper and lower portions 12, 14 of the device in a position of maximum desired extension. While in this preferred embodiment the angle of extension between the upper portion and the lower portion is about 80°, it is to be distinctly understood that the angle of extension is not limited thereto but may also be variously configured and embodied to a variety of angles depending upon the desires of the healthcare practitioner related to the party at issue. In addition, while this type of locking feature is shown in this preferred embodiment, it is to be understood that the invention is not limited thereto but may be variously embodied to hold the upper and lower portions of the device in a desired arrangement depending upon the necessities and desires of a user.

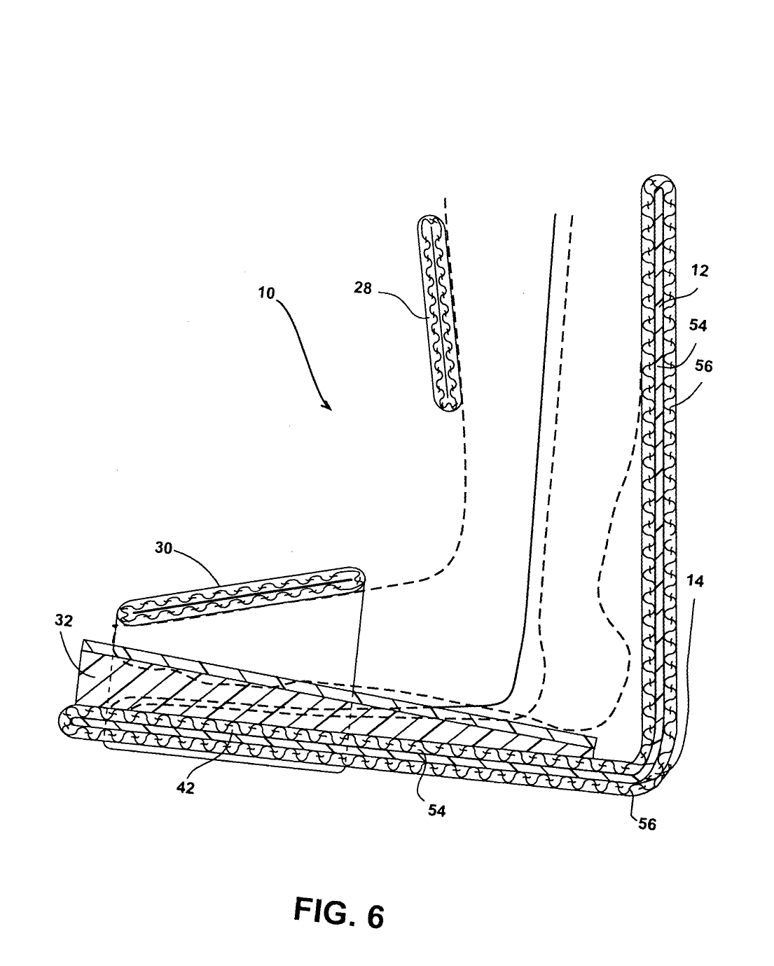

Referring now to FIG. 6, a cutaway side view of the ankle foot orthosis 10 is shown to advantage. Ankle foot orthosis 10 consists of an upper portion 12 pivotally connected to a lower portion 14 through a hinge (shown in previous figures). In this view, the upper portion 12 is configured such that it extends up the backside or posterior side of the patient's leg. The lower portion 14 extends down and around the heel to a substantially flat foot bed 42. In the preferred embodiment, the upper and lower portion members 12, 14 are configured to be foldable from a compact first position to a generally upright second position. FIG. 6 shows the present invention in this second embodiment. To further increase the portability of the present invention, the upper section 12 is configured to have a length generally shorter than the length of the lower section 14.

FIG. 6 also shows to advantage the inner layer 54 and the outer layer 56 of the fabric covering 24, as well as the removable wedge foot bed insert 32. The fabric covering 24 is made of a foam-lined fabric, but any cushioning material can be utilized. For convenience of the user, the fabric covering may be variously modified and covered so as to cover, protect, and guard the portions of a patient's leg that may contact the upper 12 and lower 14 portions of the device. In this preferred embodiment, the fabric covering 24 has an inner layer and an outer layer that are connected to each other along a top edge and along two side edges to form a first inner pocket configured to receive the upper portion 12 therein. Fabric covering 24 also has another pocket configured to receive the lower portion 14 therein. An opening in fabric covering 24 is also generally accessible through an opening between the two pockets. In the preferred embodiment, a closing flap may be utilized to cover the opening.

Fabric cover 24 may be placed over the upper and lower portions 12,14 by folding the upper portion 12 toward the lower portion 14, and pivoting these portions around one another about the hinge 16. With these two portions 12, 14 in close proximity, portions of the device 12, 14 can be pushed through the opening and configured so that the upper and lower portions of the device 12, 14 can be slipped into the pockets of the fabric cover and placed around the upper and lower portions 12, 14 of the device, and the opening then closed by a flap or other material.

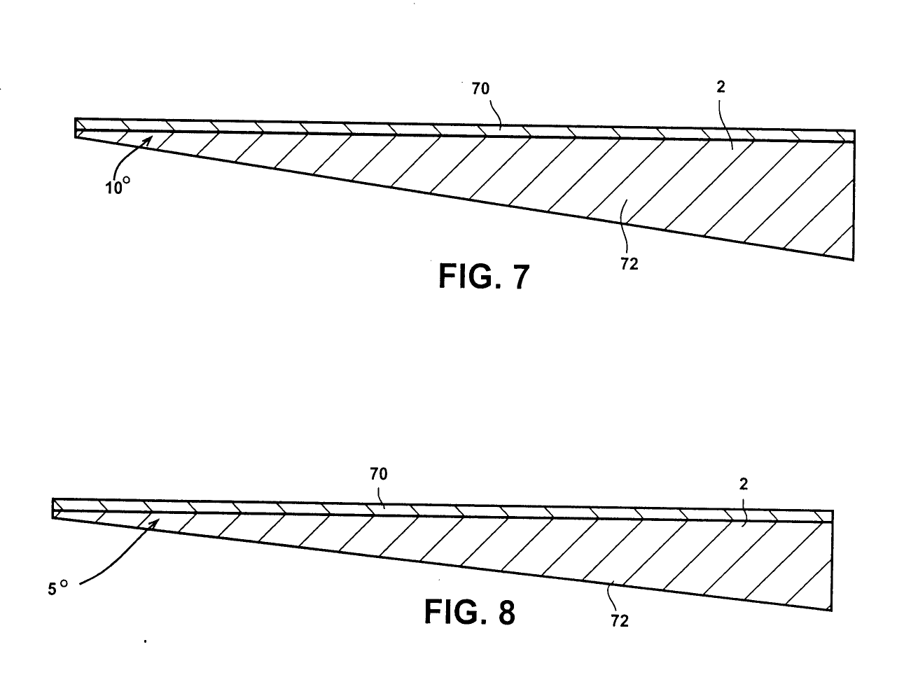

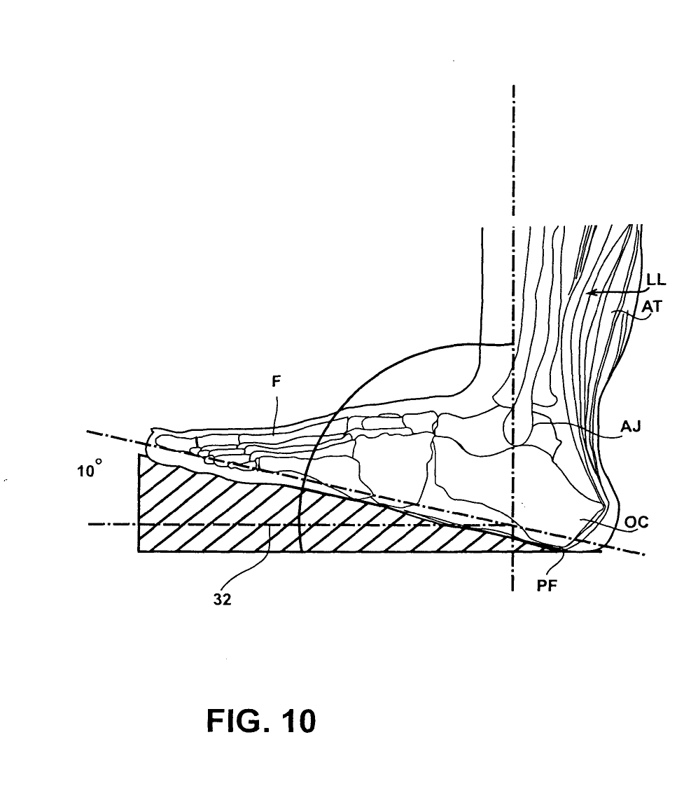

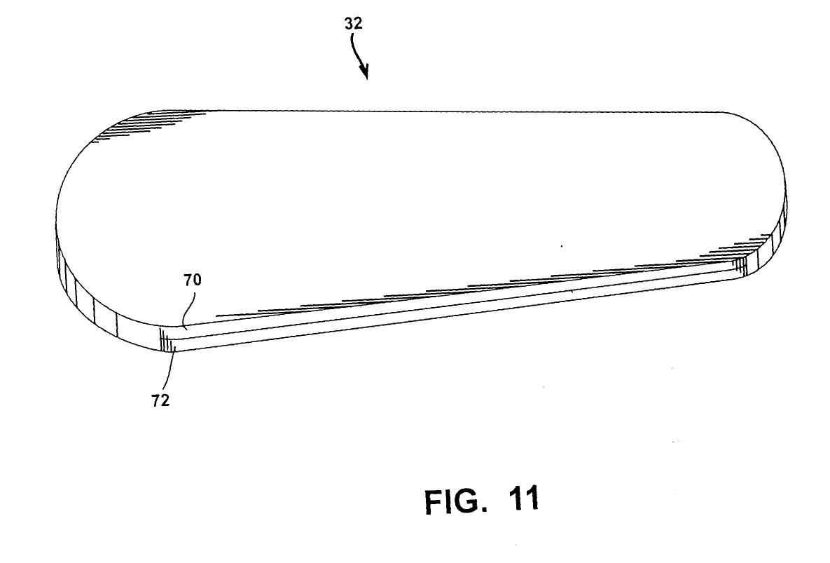

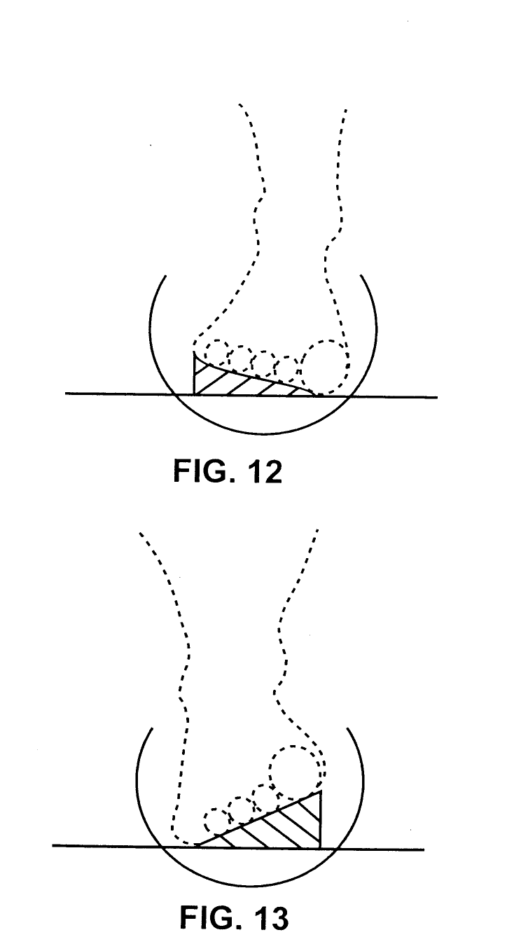

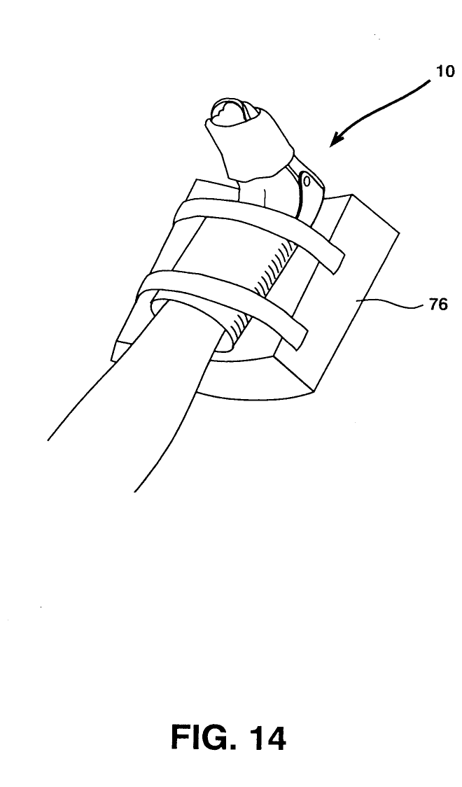

The removable wedge foot bed inserts 32 can be configured to have various features as required to achieve the desired degree of dorsiflexion or plantar flexion of the foot. The removable wedge foot bed insert 32 can also be used to cause inversion or eversion of the foot according to the particular needs of a party. To achieve inversion or eversion, the removable wedge foot bed may be configured and modified as shown in FIGS. 7, 8, 11, 12, and 13. The use of such devices are shown in FIGS. 10 and 14. FIG. 6 also shows the placement and cross-sectional structure of the lower leg attachment device 28 and the foot attachment device 30.

FIGS. 7 and 8 show two variations of removable wedge foot bed inserts 32. FIG. 7 shows a 10° wedge for use with ankle foot orthosis 10 and FIG. 8 shows a 5° wedge for use with ankle foot orthosis 10.

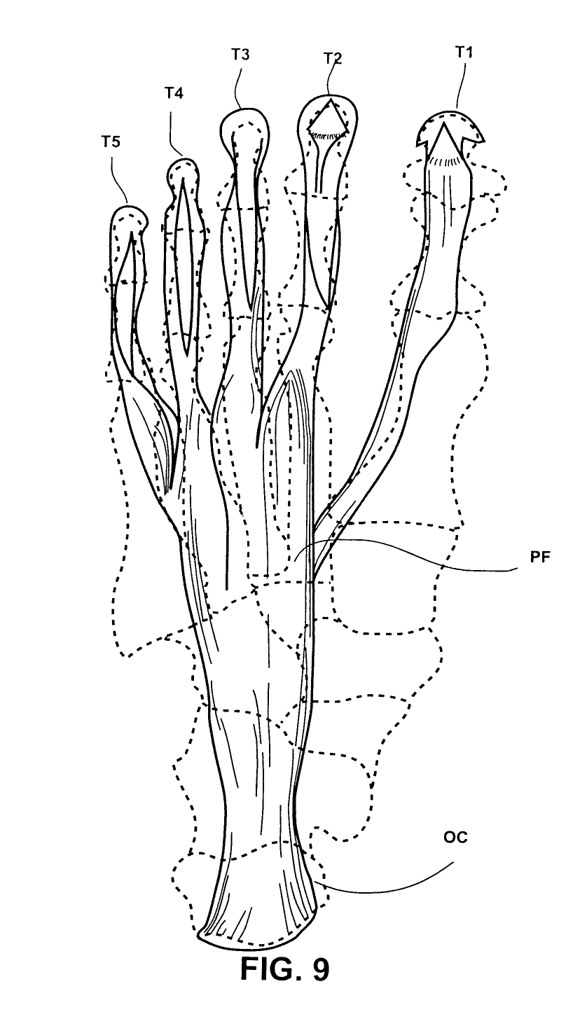

FIG. 9 shows to advantage a bottom view, or plantar view, of the human foot depicting the plantar facia PF attaching at the heel bone, or os calcis, extending longitudinally across the bottom of the foot, and eventually dividing near the heads of the metatarsal bones into five processes, with one process attaching to each of the five toes, T1 through T5.

FIG. 10 shows ankle joint AJ formed by the articulation of foot F with lower leg LL, specifically the articulation of the tibia and the fibula, the two bones which comprise the skeletal frame of the lower leg and the astragalus, the largest of the tarsal bones located next to the os calcis. FIG. 10 shows ankle joint AJ in 10° dorsiflexion. The dorsiflexion in this instance is caused by removable wedge foot bed insert 32 having a 10° incline. Use of this wedge foot bed 32 in conjunction with the other portions of the orthotic 32 results in a foot bed which is positioned 80° in relation to the angle of the upper section 12 of the orthosis 32.

FIG. 10 also shows the attachment of the plantar facia to the inner tubercle of the os calcis OC and the plantar facia PF extended slightly by the dorsiflexion of the ankle joint AJ. FIG. 10 also shows Achilles tendon AT.

In use, initially, a choice of incremental size of ankle foot orthosis 10 is made selecting a size which most closely conforms to the patient's foot and leg size. The present configuration of ankle foot orthosis 10 has sizes pediatric, small, medium, large, and extra large, which correspond to men's and women's shoe sizes as shown below:

| | |
|---|---|
| Pediatrics: | Women's: smaller than 4 |
| | Men's: smaller than 7 |
| Small: | Women's: 4-6 |
| | Men's: 7-9 |
| Medium: | Women's: 6-8 |
| | Men's: 9-11 |
| Large: | Women's: 8-10 |
| | Men's: 11-13 |
| Extra Large: | Women's: 11 and larger |
| | Men's: 14 and larger |

Next, referring to FIGS. 1 through 10, a removable wedge foot bed insert 32 of a desired angle is chosen and inserted into the foot bed 42 of the ankle foot orthosis 10. The chosen wedge 32 can be used to cause dorsiflexion or plantar flexion of the foot, and can also result in inversion or eversion of the foot in relation to the leg. A patient's lower leg LL and foot F are placed into the ankle foot orthosis 10 so that the foot F rests comfortably on the soft top layer 70 of the chosen removable wedge foot bed insert 32. Lower leg attachment strap 28 is passed from one side of upper portion 12 to the other side, across the lower leg LL. Similarly, the foot attachment strap 30 is secured across the patient's foot F. After securing the patient's foot, the patient's heel is in a floating position, and is not touching the flat foot bed 42 or the removable wedge foot bed insert 32. This floating heel position is maintained by the shape of the sagittal concavity 24 and the size of orthosis 20 selected for the patient. The positioning of the heel is further assisted by the rear heel cup 40 that is configured within the lower portion 14 of the device. This heel cup 40 may be configured to be variously shaped and to hold a variety of supportive pieces therein.

Fabric covering 24 of the orthosis 10 is designed to pad the patient's foot from any possible pressure points on the inside of the portions 12, 14. Additionally, the portions 12, 14 are shaped to minimize any possible pressure points. The fabric covering 24 also protects the collateral leg of the patient from being bumped or bruised by contact with the outside of the ankle foot orthosis 10.

The portions 12, 14 are designed to closely follow the anatomical contours of the patient's foot, ankle, and lower leg. This serves two purposes: one is to reduce the number of pressure points on the patient's foot. The other is to use the shape of the orthosis to position the patient's heel in a floating position. Since many patients being treated for plantar facitis may have tender regions on the heel bone or even bone spurs, it is important that any pressure placed on the foot, ankle and lower leg avoid pressure to the heel, while delivering even and comfortable pressure to other parts of the foot, ankle, and lower leg. Pressure must be applied to the front portion of the foot, but not the heel, so that the foot is pressed and held in a dorsiflexed position during sleep.

When ankle foot orthosis 10 is secured to an individual's lower leg and foot as described hereinabove, the ankle joint is preferably placed in dorsiflexion, but certain conditions require the use of plantar flexion, inversion, eversion, or neutral orientation, and these positions are achieved by selecting the pitch of the removable wedge foot bed inserts 32 that are used. A range of dorsiflexion of greater than 0 and inclusive of 15 has proven to be an optimal range for treatment of plantar facitis. When the ankle is so flexed, plantar facia PF and Achilles tendon AT are extended and held in a position of extension so long as the ankle foot orthosis 10 is worn as described herein.

Plantar flexion can be preferred after foot or tendon surgery, as an acclimatization to gradual stretching and lengthening of the Achilles tendon and plantar fascia by gradually decreasing plantar flexion and increasing dorsiflexion.

In another preferred embodiment, the ankle foot orthosis 10 is used in conjunction with a stabilizing cradle 76. Stabilizing cradle 76 is a device to which the ankle foot orthosis is attached and secured, as shown in FIG. 14. This mode of operation is indicated for patients recovering from hip replacement surgery or other procedures in which the hip and leg need to be immobilize. With the patient on his/her back, and one or both legs secured in an ankle foot orthosis 10, which is itself secured to a stabilizing cradle 76, the leg(s) is immobilized and the hip joint can heal optimally.

Inversion or eversion of the foot may also be desired and achieved by the use of appropriately shaped removable wedge foot bed inserts 32. This may occur after surgery on tendons in the foot. If the tendons worked on are on the medial side of the foot, it is desirable for the foot to be held in an inverted position, with the plantar surface facing toward the midline of the body. This relieves strain on the affected tendons. If the tendons worked on are on the lateral side of the foot, an everted position is desirable. A treatment of gradually changing the angle of the wedges from inverted or everted to neutral, and then gradually decreasing the plantar flexion and then increasing the dorsiflexion can be preferentially selected by physicians.

While there is shown and described the present preferred embodiment of the invention, it is to be distinctly understood that this invention is not limited thereto but may be variously embodied to practice within the scope of the following claims. From the foregoing description, it will be apparent that various changes may be made without departing from the spirit and scope of the invention as defined by the following claims.

1. A device for treating plantar facitis which comprises:
a foldable orthotic having an upper section and a lower section, each section having generally U-shaped cross-sectional profile, said upper and lower sections adjustably pivotally interconnected at a hinged portion and configured to be selectively arranged between a folded position and an extended position, said upper section configured to generally conform to the lower portion of a human leg, said lower section configured to receive a bottom surface of a foot attached to said leg;
a removable wedge foot bed insert being shaped and sized to be received in said lower section, said wedge configured to be inclined from a heel portion of said lower section to a toe portion of said lower section thereby forming an inclined foot bed which prevents plantar flexion; and
a securing mechanism configured to secure said upper portion to a lower posterior portion of a leg and to secure said lower portion to a portion of a foot, said securing mechanism flexible in at least one area above said lower portion so as to allow for adjustable degrees of dorsiflexion while preventing plantar flexion past said inclined foot bed.

2. The device of claim 1 wherein said upper portion extends from said lower section at an angle of less than 90° when in upper portion and said foldable orthotic is fully extended.

3. The device for treating plantar facitis as described in claim 1, wherein the removable wedge foot bed insert results in a foot bed which is less than 90° in relation to the upper portion.

4. The device for treating plantar facitis as described in claim 1, wherein the lower portion is at an adjustable angle of less than 90° to the upper portion and the removable wedge foot bed insert results in a foot bed which is 85° in relation to the upper portion.

5. The device for treating plantar facitis as described in claim 1, wherein the lower portion is at an adjustable angle of less than 90° to the upper portion and the removable wedge foot bed insert results in a foot bed which is 80° in relation to the upper portion.

6. The device for treating plantar facitis as described in claim 1, wherein the lower portion is at an adjustable angle of less than 90° in relation to the upper portion and the removable wedge foot bed insert results in a foot bed which may vary from 90° to 75° in relation to the upper portion.

7. The device for treating plantar facitis as described in claim 1 wherein the portion is selected from a variety of sizes of portion having incremental sizing, in which the variety of incremental sizing corresponds to any standard system of shoe sizes.

8. An ankle-foot orthosis comprising:
a foldable orthotic having an upper portion selectively pivotally connected to a lower portion the upper and lower sections each having a generally U-shaped cross-sectional configuration, and configured to fold upon one another in a first position and to extend to second position, a lower portion attached to and extending away from the upper section said lower section having a foot bed and a generally U-shaped cross-sectional configuration; and
a removable wedge foot bed insert configured for placement on to the flat foot bed in the lower portion arm which when placed on the lower portion when the lower portion is at substantially a right angle to the upper portion, presents a foot bed in which the medial side of the foot bed is lower than the lateral side of the foot bed, and the cross-section of the foot bed from one side to the other shows a top surface angle of between 0° and 15°.

9. The ankle-foot orthosis of claim 8 wherein the upper portion has a length that is proportionally less than the length of the lower portion.

10. An ankle-foot orthosis comprising:
a foldable shell, having an upper section pivotally connected to a lower section, both upper and lower sections having generally U-shaped cross-sections configured to extend at an angle of less than 90° from said upper section when the upper and lower sections are fully extended, the lower section also having a generally flat foot bed portion, said generally flat foot bed portion having a heel portion and a toe portion, said heel portion narrower than said toe portion and designed for close anatomical fit with a heel of a human patient, said upper section configured for close and anatomically conforming shape with a lower posterior portion of a human leg, said upper section also having a concavity which conforms to said human leg and maintains a human heel in a floated position from said flat foot bed, said upper section extending along a length to a location below the thickest portion of the gastrocsoleus muscles of a patient, the lower section being configured to receive a removable wedge foot bed insert, and said foldable portion being shaped to define an opening around a medial and a lateral prominence of a human ankle;
a removable wedge foot bed insert, said insert shaped and sized to be received in said foot bed portion, said wedge being inclined from a heel portion of the foot bed to a toe portion of the foot bed thereby forming an inclined foot bed which prevents plantar flexion, said the removable wedge foot bed insert made of a semi-rigid material and having a cushioning top surface;
a soft jacket having a securing means attached, said soft jacket configured to cover said inside and outside surfaces of said portion, and to connect with a securing means; and
a securing mechanism said foldable portion to the lower posterior portion of the leg and foot, said securing mechanism being flexible in at least an area above said foot bed to allow for adjustable degrees of dorsiflexion while preventing plantar flexion past the inclined foot bed.

11. The orthosis of claim 10, wherein said lower portion is at an angle of less than 90° to said upper portion when said foldable portion is fully extended.

12. The orthosis of claim 11, wherein the addition of the removable wedge foot bed insert results in a foot bed surface which is 85° in relation to the upper portion.

13. The orthosis of claim 11, wherein the removable wedge foot bed insert results in a foot bed which is 80° in relation to the upper portion.

14. The orthosis of claim 11, wherein the addition of the removable wedge foot bed insert results in a foot bed which may vary from less than 90° to 75° in relation to the upper portion.

15. The orthosis of claim 11, which further comprises a stabilizing, cradle to which one or two orthosis can be attached, and which immobilizes said orthosis.
